(19) United States
(12) Patent Application Publication
(10) Pub. No.: US 2026/0177651 A1
(43) Pub. Date: Jun. 25, 2026

(54) METHODS FOR QUANTIFYING N-ACETYLCYSTEINE AND GLUTATHIONE IN BRAIN TISSUE USING MAGNETIC RESONANCE SPECTROSCOPY

(71) Applicants: NeuroNasal, Inc., Wexford, PA (US); Cornell University, Ithaca, NY (US)

(72) Inventors: Douglas A. Greene, Basking Ridge, NJ (US); Dikoma C. Shungu, Roosevelt Island, NY (US)

(21) Appl. No.: 19/430,503

(22) Filed: Dec. 23, 2025

Related U.S. Application Data
(60) Provisional application No. 63/737,849, filed on Dec. 23, 2024.

(51) Int. Cl. G01R 33/56; A61B 5/00; A61B 5/055

(52) U.S. Cl. CPC G01R 33/5601; A61B 5/0042; A61B 5/055

(57) ABSTRACT

A method for separately quantifying N-acetylcysteine and glutathione in brain tissue includes administering N-acetylcysteine to a subject, acquiring a first magnetic resonance spectroscopy signal from a region of brain tissue of the subject, the first magnetic resonance spectroscopy signal comprising a cysteine β-proton signal, acquiring a second magnetic resonance spectroscopy signal from the region of brain tissue, the second magnetic resonance spectroscopy signal comprising an N-acetyl proton signal, and determining a concentration of N-acetylcysteine in the region of brain tissue based on the N-acetyl proton signal and determining a concentration of glutathione in the region of brain tissue based on a difference between the cysteine β-proton signal and the N-acetyl proton signal.

### CROSS-REFERENCE TO RELATED APPLICATIONS

This application claims priority to U.S. Ser. No. 63/737,849 filed on Dec. 23, 2024, titled METHODS OF QUANTIFYING N-ACETYLCYSTEINE, N-ACETYLCYSTEINE DERIVATIVES AND ACTIVE METABOLITES IN THE BRAIN, which is hereby incorporated by reference in its entirety.

### FIELD OF INVENTION

The present disclosure relates to magnetic resonance spectroscopy techniques for analyzing brain metabolites, and more particularly to methods for separately quantifying N-acetylcysteine and glutathione concentrations in brain tissue to optimize therapeutic dosing regimens for neurological disorders.

### BACKGROUND

N-acetylcysteine (NAC) is a precursor of L-cysteine that results in glutathione (GSH) elevation through biosynthesis. NAC has therapeutic applications for various central nervous system (CNS) disorders.

Magnetic resonance spectroscopy (MRS) is a non-invasive analytical technique that can detect and measure metabolic changes in the brain. NAC and GSH share common molecular features that produce overlapping MRS resonance peaks. When NAC is administered as a therapeutic agent, conventional MRS techniques cannot distinguish between signals emanating from GSH and signals emanating from NAC itself.

The therapeutic relevance of NAC versus NAC-derived GSH may differ depending on the specific brain disorder being treated. Accordingly, there is a need for methods that can separately quantify NAC and GSH concentrations in brain tissue to optimize therapeutic dosing regimens.

### BRIEF DESCRIPTION OF FIGURES

The present disclosure will be more readily understood from a detailed description of some example embodiments taken in conjunction with the following figures:

FIG. 1 depicts a diagram illustrating functions of N-acetylcysteine in glutathione synthesis by glutathione-depleted red blood cells.

FIG. 2 depicts molecular structures of glutathione and N-acetylcysteine.

FIG. 3 depicts a magnetic resonance spectroscopy spectrum for glutathione.

FIG. 4 depicts a magnetic resonance spectroscopy spectrum for N-acetylcysteine.

FIG. 5 depicts magnetic resonance spectroscopy spectra demonstrating a subtraction approach for quantifying and differentiating brain levels of N-acetylcysteine from glutathione, according to aspects of the present disclosure.

FIG. 6 depicts a diagram illustrating mechanisms of action of N-acetylcysteine.

FIG. 7 depicts edited and unedited magnetic resonance spectroscopy spectra for N-acetylcysteine and glutathione, according to aspects of the present disclosure.

### DETAILED DESCRIPTION

Various non-limiting embodiments of the present disclosure will now be described to provide an overall understanding of the principles of the structure, function, and use of the systems, apparatuses, devices, and methods disclosed. One or more examples of these non-limiting embodiments are illustrated in the accompanying figures. Those of ordinary skill in the art will understand that systems, apparatuses, devices, and methods specifically described herein and illustrated in the accompanying drawing are non-limiting embodiments. The features illustrated or described in connection with one non-limiting embodiment may be combined with the features of other non-limiting embodiments. Such modifications and variations are intended to be included within the scope of the present disclosure.

The systems, apparatuses, devices, and methods disclosed herein are described in detail by way of examples and with reference to the figures. The examples discussed herein are examples only and are provided to assist in the explanation of the apparatuses, devices, systems and methods described herein. None of the features or components shown in the drawings or discussed below should be taken as mandatory for any specific implementation of any of these apparatuses, devices, systems or methods unless specifically designated as mandatory. For ease of reading and clarity, certain components, modules, or methods may be described solely in connection with a specific figure. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

N-acetylcysteine (NAC) is a precursor of L-cysteine that results in glutathione (GSH) elevation through biosynthesis. NAC is a powerful antioxidant that acts directly as a scavenger of free radicals, including oxygen free radicals. NAC may be used as a treatment option for disorders resulting from the generation of free oxygen radicals. NAC has a range of pleotropic salutary effects on acute and chronic central nervous system (CNS) disorders.

NAC demonstrates multiple beneficial effects in treating both acute and chronic disorders of the CNS. These therapeutic benefits operate through several distinct biochemical and pharmacological mechanisms of action. NAC functions as an antioxidant by neutralizing reactive oxygen species (ROS) and chelating oxidative reactive metal ions, thereby protecting cellular components from oxidative damage. NAC also exhibits anti-inflammatory properties in the CNS. Furthermore, NAC serves as a precursor molecule, providing cysteine for the de-novo synthesis of the antioxidant GSH and the modulation of neural signaling through the cystine-glutamate antiporter system. Many of the properties attributed to NAC are also shared by GSH and cysteine, which are themselves NAC derivatives.

Magnetic resonance spectroscopy (MRS) is a technique associated with magnetic resonance imaging (MRI). MRS, also known as nuclear magnetic resonance (NMR) spectroscopy, is a non-invasive, ionizing-radiation-free analytical technique that may detect and measure metabolic changes in an organ, such as the brain. In some cases, MRS may be used to acquire a signal from a single localized region of the brain, referred to as a voxel. In some cases, MRS may be used to determine a relative concentration of a biochemical in a region of the brain. In some cases, MRS may be used to determine a physical property of a region of the brain. In some cases, MRS may be used to delineate the route, rate, and longevity by and with which exogenously administered NAC is transported, transformed into, and persists as intracranial active metabolites.

Conventional MRS measurement of GSH presents a challenge when the active pharmaceutical ingredient is NAC rather than GSH itself. Because of multiple overlapping peaks from other metabolites, conventional MRS quantitation of GSH is based on quantification of the visually detectable GSH cysteine β-proton resonating at approximately 2.95 ppm. Both GSH and NAC possess the same β-cysteinyl proton groups. The α-CH and β-CH₂ groups of the cysteine moieties of each molecule produce overlapping MRS signals. As a result, conventional MRS measures the total amount of NAC and GSH rather than GSH alone. Furthermore, conventional MRS does not distinguish between these two molecular species that have distinct and largely non-overlapping pharmacological properties relevant to the treatment of brain disorders such as traumatic brain injury (concussion) and Parkinson's disease, among others.

This distinction between NAC and GSH is not relevant when the pharmaceutical agent administered is GSH, where a rise in the MRS signal for GSH is reflective of a bona fide rise in the GSH content of the brain. This distinction, however, becomes relevant when the administered pharmaceutical agent is NAC, following which the conventional MRS measurement of GSH is flawed because the conventional MRS measurement does not distinguish between GSH and NAC. Hence, conventional MRS measures the increase in the sum of GSH and NAC, rather than measuring the accumulation of GSH or NAC individually. Measuring GSH or NAC individually is useful to fully characterize and define the dose-related pharmaceutical properties of the administered NAC.

The methods described herein address this problem by providing approaches that differentiate between a post-dose rise in GSH or NAC by measuring each moiety individually. In some cases, the MRS analytics may be augmented to measure changes in NAC or GSH, rather than the summation of the two molecules, to optimize dosing of NAC to treat various brain diseases. In some cases, editing the MRS analytics following administration of pharmaceutical quantities of NAC may use simultaneous measurements of changes in the MRS spectra of both the cysteine β-proton and the N-acetyl proton to measure changes in NAC. The N-acetyl proton signal may be superimposed on that of N-acetyl aspartate (NAA). Changes in the cysteine β-proton alone without concurrent increase in the N-acetyl proton may be indicative of a change in GSH content following NAC administration. Thus, a subtraction approach in accordance with the present disclosure may separately measure and detect changes in GSH or NAC following administration of NAC.

The methods described herein provide approaches for quantifying NAC, NAC derivatives, and active metabolites in human brain tissue, enabling precise optimization of NAC dosing regimens. By measuring these compounds' concentrations and understanding their pharmacokinetics in brain tissue, clinicians may make evidence-based decisions regarding dosage amounts and intervals between doses. This optimization may maximize therapeutic efficacy while minimizing potential side effects in the treatment of various brain disorders. The described methods advance the personalization of NAC therapy and may improve treatment outcomes for patients with neurological and psychiatric conditions. Furthermore, these quantification techniques may serve as tools for future research into NAC's mechanisms of action and the development of enhanced therapeutic protocols.

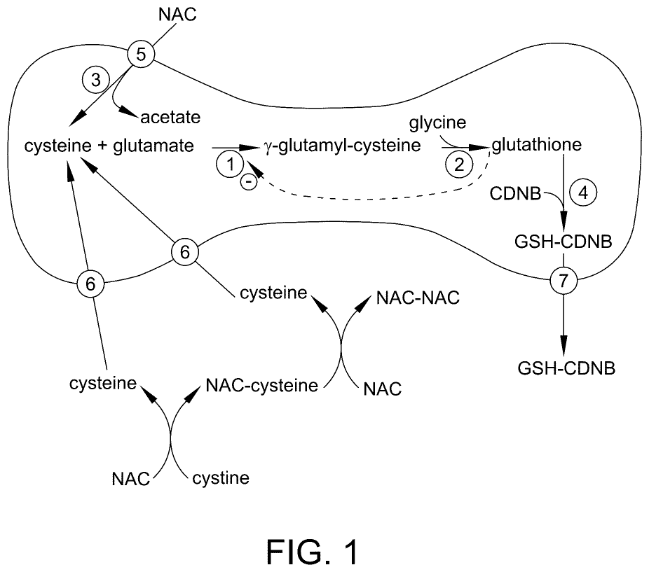

Referring to FIG. 1, a diagram illustrates the functions of NAC in glutathione synthesis by GSH-depleted red blood cells. FIG. 1 depicts a cell membrane boundary containing intracellular and extracellular compartments with various biochemical pathways and reactions.

Inside the cell, cysteine and glutamate may combine through a γ-glutamyl-cysteine synthesis reaction (1) to form γ-glutamyl-cysteine. The γ-glutamyl-cysteine synthesis reaction (1) may exhibit feedback inhibition, as indicated by a dashed line in FIG. 1. The γ-glutamyl-cysteine may then combine with glycine through a glutathione synthesis reaction (2) to produce glutathione. In some cases, glutathione may react with CDNB (1-chloro-2,4-dinitrobenzene) through a GSH-CDNB conjugation reaction (4) to form GSH-CDNB. The GSH-CDNB may then be exported from the cell via a GSH-CDNB export pathway (7).

With continued reference to FIG. 1, NAC may enter the cell and undergo a NAC deacetylation reaction (5) to release acetate and contribute to a cysteine production reaction (3). A cysteine transport pathway (6) may facilitate the movement of cysteine into the cell from extracellular sources. Outside the cell, NAC may participate in thiol-disulfide exchange reactions with cystine, NAC-cysteine, and NAC-NAC. These extracellular reactions may release cystine-derived cysteine, which may enter the cell through the cysteine transport pathway 6.

Approximately 95% of NAC-derived cysteine substrate for intracellular GSH synthesis may not be derived from carrier-mediated entry and subsequent enzymatic deacetylation of NAC through the NAC deacetylation reaction (5) and the cysteine production reaction (3). In contradistinction, approximately 95% of NAC-derived cysteine may reflect NAC's ability to cleave plasma cystine (oxidized cysteine disulfide) residues extracellularly, releasing cystine-derived cysteine which enters the cell through the cysteine transport pathway (6) to promote GSH synthesis via the γ-glutamyl-cysteine synthesis reaction (1) and the glutathione synthesis reaction (2). Were the blood-brain barrier (BBB) or other portals of entry to the brain to function similarly to the erythrocyte plasma membrane with regard to NAC transport, then the bulk of NAC-derived cysteine entering the brain parenchyma would in fact have been derived from circulating or extra-cranial cystine rather than administered NAC.

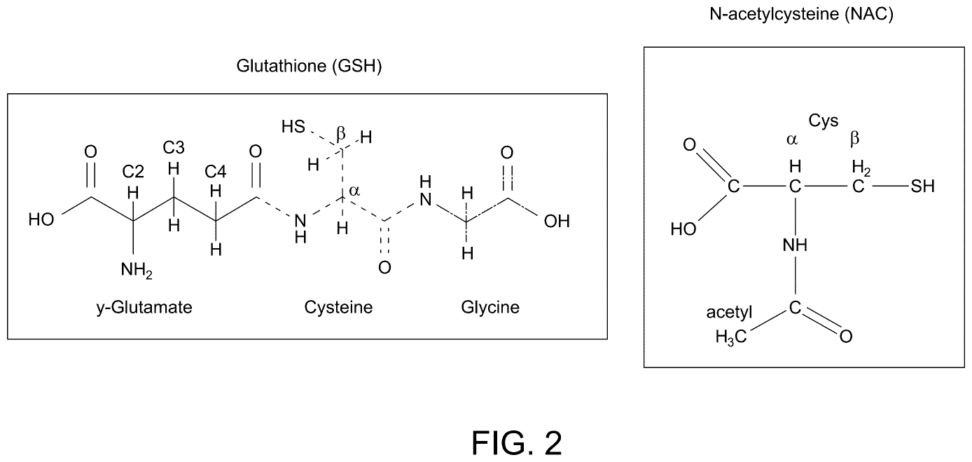

Referring to FIG. 2, the molecular structures of GSH and NAC are depicted. GSH is a tripeptide composed of three amino acid residues: γ-Glutamate, Cysteine, and Glycine. The γ-Glutamate portion of GSH contains a carboxylic acid group, an amino group, and carbon atoms. The Cysteine portion of GSH includes an α-carbon, a β-carbon with hydrogen atoms, and a thiol group (HS). The Glycine portion terminates the GSH molecule with a carboxylic acid group.

As further shown in FIG. 2, N-acetylcysteine is a simpler molecule consisting of a cysteine residue with an acetyl group attached to the nitrogen. The cysteine portion of NAC includes an α-carbon, a β-carbon bearing a thiol group (SH), and a carboxylic acid group. The acetyl moiety of NAC contains a methyl group (H₃C) bonded to a carbonyl carbon. Both glutathione and N-acetylcysteine share common structural features in their cysteine-based α-CH and β-CH₂ groups. These shared structural features are relevant to the magnetic resonance spectroscopy signatures of both molecules. The α-CH and β-CH₂ groups of the cysteine moieties of glutathione and N-acetylcysteine produce overlapping MRS resonance peaks. As a result of this structural similarity, conventional MRS techniques may be unable to distinguish between glutathione and N-acetylcysteine based on the cysteine β-proton signal alone. The acetyl moiety present in N-acetylcysteine but absent in glutathione provides a distinguishing structural feature that may be exploited in MRS analysis to differentiate between the two compounds.

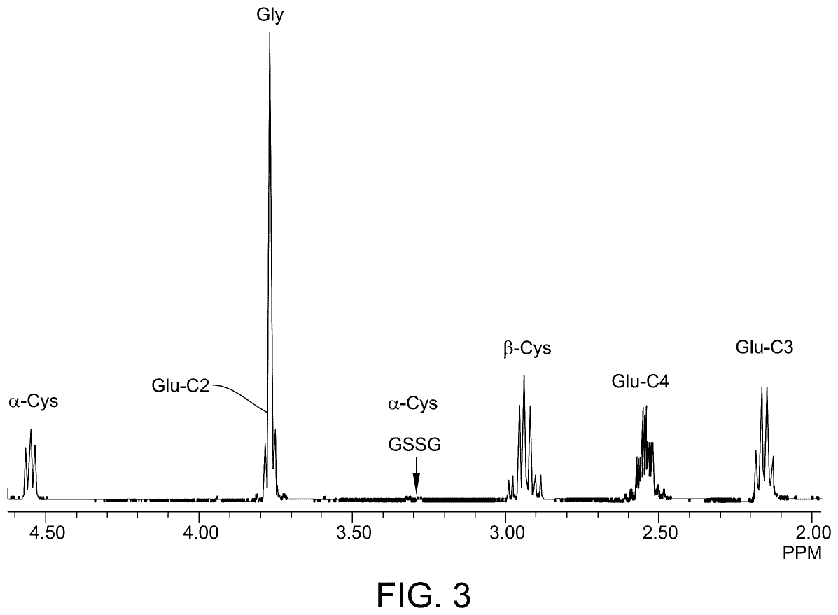

Referring to FIG. 3, a magnetic resonance spectroscopy spectrum for GSH is depicted. The spectrum displays signal intensity along the vertical axis and chemical shift measured in parts per million (ppm) along the horizontal axis, ranging from approximately 2.00 ppm to 4.50 ppm. The spectrum shown in FIG. 3 displays several labeled resonance peaks corresponding to different proton groups within the glutathione molecule. A peak labeled Gly corresponds to the glycine residue and appears near 3.75 ppm. Peaks labeled Glu-C2, Glu-C3, and Glu-C4 correspond to protons at different carbon positions of the glutamate residue. The Glu-C2 peak appears near 3.75 ppm, the Glu-C4 peak appears near 2.50 ppm, and the Glu-C3 peak appears near 2.15 ppm.

With continued reference to FIG. 3, the cysteine residue of glutathione produces peaks labeled α-Cys and β-Cys. The α-Cys peaks appear near 4.50 ppm and 3.50 ppm. The β-Cys peak appears near 2.95 ppm. An additional marker labeled GSSG indicates the position where oxidized glutathione disulfide would appear in the spectrum near 3.30 ppm.

As further shown in FIG. 3, conventional MRS quantitation of GSH may be based on quantification of the visually detectable GSH cysteine β-proton resonating at approximately 2.95 ppm. This β-Cys peak at approximately 2.95 ppm serves as the basis for conventional GSH measurement because multiple overlapping peaks from other metabolites may obscure other regions of the spectrum. The reliance on the β-Cys peak for GSH quantitation presents a challenge when N-acetylcysteine is administered, as N-acetylcysteine also possesses a β-cysteinyl proton group that resonates at a similar chemical shift position.

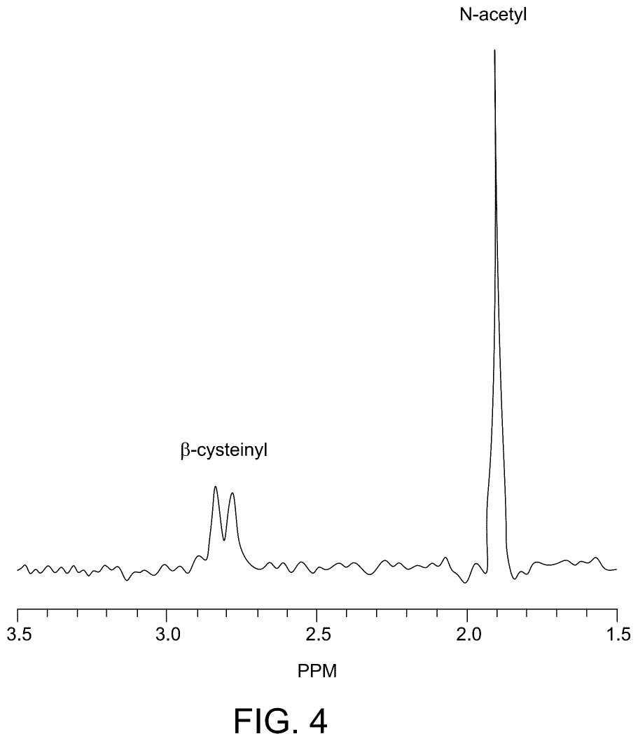

Referring to FIG. 4, a magnetic resonance spectroscopy spectrum NAC is depicted. The MRS spectrum displays chemical shift values along the horizontal axis ranging from approximately 3.5 to 1.5 parts per million (ppm). Two distinct peaks are visible in the spectrum. A peak labeled as β-cysteinyl appears at approximately 2.9 ppm, corresponding to the beta carbon protons of the cysteine moiety of NAC. A larger peak labeled as N-acetyl appears at approximately 2.0 ppm, corresponding to the methyl protons of the acetyl group attached to the nitrogen atom of NAC.

With continued reference to FIG. 4, the spectrum demonstrates the characteristic MRS signature of NAC, showing the resonance positions of the protons associated with both the cysteinyl and acetyl portions of the molecule. The β-cysteinyl peak at approximately 2.9 ppm in the NAC spectrum overlaps with the β-Cys peak of glutathione shown in FIG. 3, which also appears near 2.95 ppm. This overlap between the β-cysteinyl peaks of NAC and GSH is the source of the measurement problem addressed by the methods described herein. Conventional MRS techniques that rely on the β-cysteinyl peak for quantitation may be unable to distinguish between NAC and GSH contributions to the signal at this chemical shift position.

As further shown in FIG. 4, the N-acetyl peak at approximately 2.0 ppm provides a distinguishing spectral feature that is present in NAC but absent in glutathione. This N-acetyl peak corresponds to the methyl protons of the acetyl moiety that is attached to the nitrogen atom of NAC. Because glutathione lacks an acetyl group, glutathione does not produce a corresponding peak at this chemical shift position. The presence of the N-acetyl peak in the NAC spectrum may be exploited to differentiate NAC from GSH in MRS measurements, as described in the methods disclosed herein.

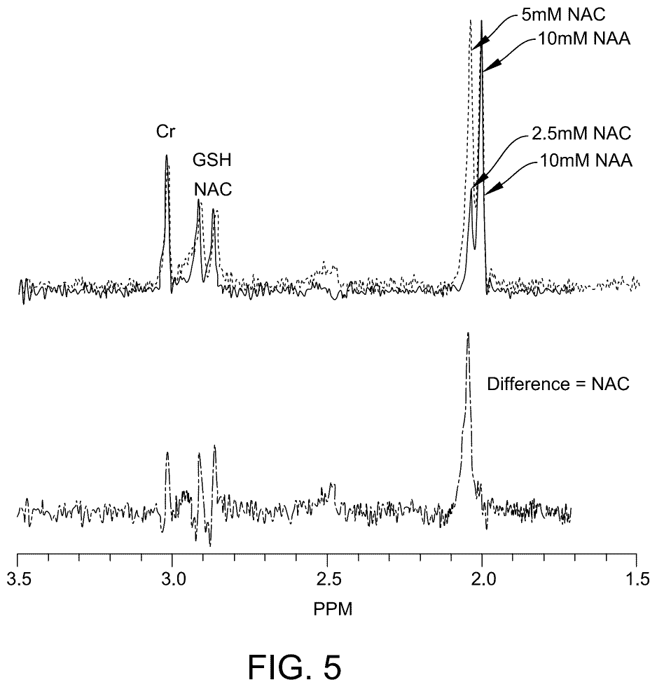

Referring to FIG. 5, magnetic resonance spectroscopy spectra demonstrate an approach for quantifying and differentiating brain levels of N-acetylcysteine from glutathione. The upper portion of FIG. 5 shows two overlaid spectra plotted against a horizontal axis labeled in parts per million ranging from approximately 3.5 to 1.5 ppm. A first spectrum shown in blue corresponds to a sample containing 5 millimolar (mM)N-acetylcysteine and 10 mM N-acetylaspartate (NAA). A second spectrum shown in red corresponds to a sample containing 2.5 mM N-acetylcysteine and 10 mM NAA.

Several peaks are labeled in the upper spectra of FIG. 5. A creatine peak, a glutathione peak, and an N-acetylcysteine peak appear in the region around 3.0 ppm. A prominent peak appears near 2.0 ppm where the N-acetylcysteine and N-acetylaspartate signals overlap. At this location near 2.0 ppm, the blue spectrum shows a higher intensity than the red spectrum due to the higher N-acetylcysteine concentration in the sample corresponding to the blue spectrum. The difference in peak height at approximately 2.0 ppm between the two spectra reflects the difference in N-acetylcysteine concentration between the 5 mM and 2.5 mM samples.

With continued reference to FIG. 5, the lower portion of the figure displays a difference spectrum shown in black. This difference spectrum results from subtracting the red spectrum (2.5 mM NAC with 10 mM NAA) from the blue spectrum (5 mM NAC with 10 mM NAA). The difference spectrum is labeled as representing N-acetylcysteine. The difference spectrum shows a prominent positive peak near 2.0 ppm corresponding to the isolated N-acetylcysteine signal. Smaller residual peaks appear in other regions of the difference spectrum.

As further shown in FIG. 5, the increased NAC-derived signal from 2.5 mM to 5 mM NAC may be detected and differentiated from the background GSH signal using the difference in the N-acetyl peak to calculate and interpret the difference. The subtraction approach illustrated in FIG. 5 enables separate measurement and detection of changes in glutathione or N-acetylcysteine following administration of N-acetylcysteine.

In some cases, editing the MRS analytics following administration of pharmaceutical quantities of NAC may use simultaneous measurements of changes in the MRS spectra of both the cysteine β-proton and the N-acetyl proton. Changes in NAC may be measured by detecting changes in the N-acetyl proton signal, which may be superimposed on that of N-acetylaspartate. Changes in the cysteine β-proton alone without concurrent increase in the N-acetyl proton may be indicative of a change in GSH content following NAC administration. By comparing the magnitude of change in the cysteine β-proton signal with the magnitude of change in the N-acetyl proton signal, the contributions of NAC and GSH to the total cysteine β-proton signal may be deconvolved. This approach may allow clinicians and researchers to separately quantify NAC and GSH concentrations in brain tissue following NAC administration, thereby enabling optimization of NAC dosing regimens for various brain disorders.

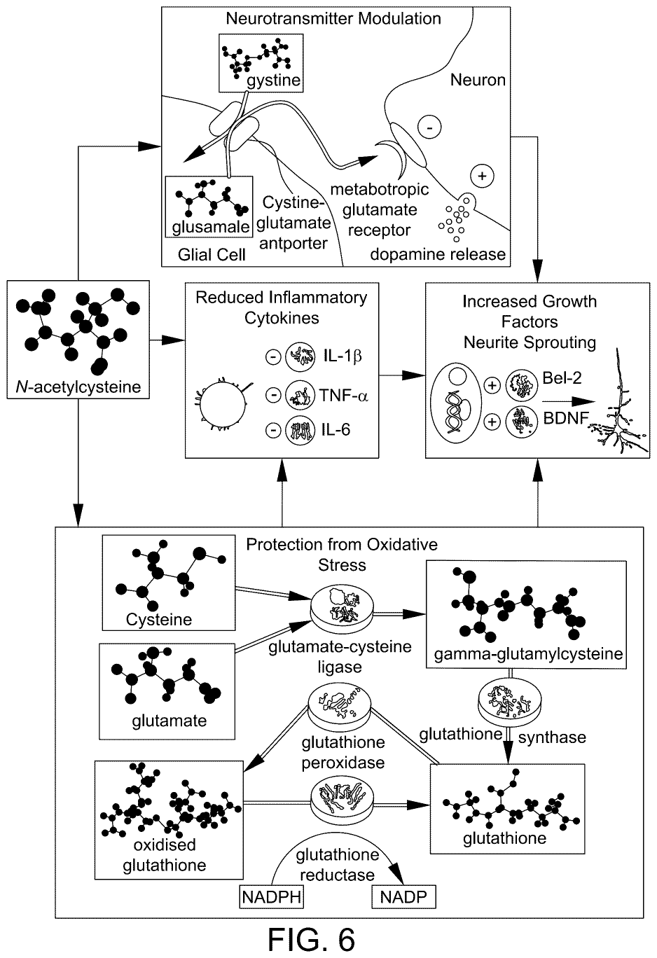

Referring to FIG. 6, a diagram illustrates the mechanisms of action of NAC. Understanding these distinct mechanisms is central to the methods described herein, as the ability to separately quantify NAC and GSH enables clinicians to target specific therapeutic pathways based on the particular brain disorder being treated. The upper panel of FIG. 6 illustrates neurotransmitter modulation via the cystine-glutamate antiporter system. This panel shows a glial cell and a neuron with a cystine-glutamate antiporter system. In some cases, cystine exchange at the antiporter may lead to glutamate release from glial cells. The released glutamate may act on metabotropic glutamate receptors located on inhibitory neurons. This action may result in inhibition of certain neuronal activity. In some cases, the cystine-glutamate antiporter activity may also facilitate dopamine release. NAC and NAC derivatives including L-cysteine and cystine may serve to release the neurotransmitter glutamate from glial cells, thereby modulating glutamate-mediated neuroexcitotoxicity. Glutamate-mediated neuroexcitotoxicity is implicated in disorders such as traumatic brain injury. This anti-glutamatergic effect is distinct from NAC's capacity as a GSH precursor, and the MRS quantification methods described herein enable clinicians to monitor NAC concentrations specifically to optimize this anti-neuroexcitotoxicity activity.

With continued reference to FIG. 6, the center-left panel depicts reduced inflammatory cytokines. This panel shows a cell with negative indicators associated with interleukin-1β (IL-1β), tumor necrosis factor-α (TNF-α), and interleukin-6 (IL-6). The negative indicators represent decreased production of these inflammatory markers. NAC may exhibit anti-inflammatory properties in the CNS through reduction of these inflammatory cytokines. This anti-inflammatory action is distinct from NAC's GSH precursor capacity, since GSH functions as a catalytic antioxidant. By separately quantifying NAC and GSH using the methods described herein, clinicians may determine whether administered NAC is present in sufficient concentrations to provide direct anti-inflammatory effects independent of GSH-mediated antioxidant activity.

The center-right panel of FIG. 6 illustrates increased growth factors and neurite sprouting. This panel shows positive indicators associated with Bcl-2 and brain-derived neurotrophic factor (BDNF). An arrow in this panel points to an image of neuronal growth and branching. In some cases, the actions of NAC may converge upon mechanisms promoting cell survival and growth factor synthesis, leading to increased neurite sprouting.

As further shown in FIG. 6, the lower panel depicts protection from oxidative stress through the glutathione synthesis pathway. This panel shows the conversion of cysteine and glutamate via glutamate-cysteine ligase to γ-glutamylcysteine. The γ-glutamylcysteine may then be converted by glutathione synthase to glutathione. The lower panel also illustrates the glutathione redox cycle. In the glutathione redox cycle, glutathione may be oxidized to oxidized glutathione via glutathione peroxidase. The oxidized glutathione may then be reduced back to glutathione via glutathione reductase. This reduction reaction uses nicotinamide adenine dinucleotide phosphate in its reduced form (NADPH), which is converted to nicotinamide adenine dinucleotide phosphate (NADP) in the process. NAC serves as a substrate for glutathione synthesis by providing cysteine for the de-novo synthesis of GSH. The MRS methods described herein, which utilize the N-acetyl proton signal to quantify NAC and the difference between the cysteine β-proton signal and the N-acetyl proton signal to quantify GSH, enable clinicians to monitor the conversion of administered NAC into GSH through this biosynthetic pathway.

With continued reference to FIG. 6, the pleotropic actions of NAC beyond serving as a GSH precursor have direct implications for the separate quantification methods described herein and for optimizing NAC dosing regimens. Because NAC and GSH have distinct and largely non-overlapping pharmacological properties as illustrated in FIG. 6, the ability to separately measure these two molecular species using the MRS techniques disclosed herein provides clinically actionable information that cannot be obtained from conventional MRS measurements of the combined NAC and GSH signal. In some cases, for disorders such as Parkinson's disease where auto-oxidation of dopamine is thought to be a source of disease-relevant oxidative stress, increased GSH content may be a relevant pharmacological target on which to design dose level and dose interval, and the methods described herein enable monitoring of GSH accumulation specifically. In some cases, for acute concussion, a relevant biomarker on which to optimize NAC dose level and dose interval may be some balance between NAC level and GSH level, to achieve dual antioxidant and anti-neuroexcitotoxicity activity as depicted in the upper and lower panels of FIG. 6. The methods described herein enable clinicians to separately measure NAC and GSH concentrations using the subtraction approach illustrated in FIG. 5, thereby allowing optimization of dosing based on the specific therapeutic targets relevant to different brain disorders as illustrated in FIG. 6.

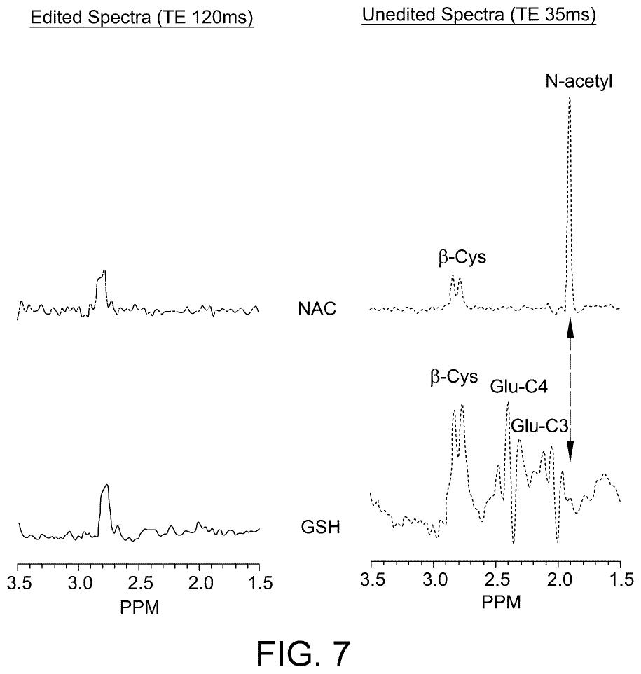

Referring to FIG. 7, edited and unedited magnetic resonance spectroscopy spectra for NAC and GSH are depicted. FIG. 7 is divided into two columns. The left column shows edited spectra acquired at an echo time (TE) of 120 milliseconds. The right column shows unedited spectra acquired at an echo time of 35 milliseconds. The top row of FIG. 7 displays the spectra for NAC, while the bottom row displays the spectra for GSH.

The edited spectra shown in the left column of FIG. 7 are acquired using specialized pulse sequences. These specialized pulse sequences may suppress unwanted signals and enhance detection of specific molecules such as glutathione. In some cases, the editing techniques may isolate signals from target metabolites that would otherwise be obscured by overlapping signals from more abundant compounds. The edited spectra for both NAC and GSH exhibit similar spectral patterns with peaks appearing in the region between approximately 2.5 and 3.0 parts per million (ppm). The similarity of the edited spectral patterns for NAC and GSH in this region reflects the shared cysteine-based structural features of both molecules.

With continued reference to FIG. 7, the unedited spectra shown in the right column are acquired at a shorter echo time of 35 milliseconds. The unedited spectra show raw, unprocessed spectral data. The shorter echo time used for the unedited spectra may provide stronger overall signal intensity compared to the longer echo time used for the edited spectra. However, the unedited spectra display more complex spectral patterns with potentially more overlapped signals from multiple metabolites.

As further shown in FIG. 7, the unedited spectra for both NAC and GSH display a prominent N-acetyl peak at approximately 2.0 ppm. In the NAC unedited spectrum, this N-acetyl peak corresponds to the methyl protons of the acetyl group attached to the nitrogen atom of NAC. A dashed arrow in the unedited spectra indicates the relationship between the N-acetyl peak positions in the NAC and GSH spectra. The β-cysteine (β-Cys) peaks are labeled in both unedited spectra and appear at approximately 2.9 ppm. The β-Cys peaks in the NAC and GSH unedited spectra demonstrate the superimposability of the cysteine β-proton peaks of GSH and NAC. This superimposability illustrates how conventional MRS measurements may be unable to distinguish between these two molecular species without spectral editing techniques.

The GSH unedited spectrum shown in FIG. 7 additionally displays labeled peaks for glutamate C4 (Glu-C4) and glutamate C3 (Glu-C3) in the region between approximately 2.0 and 2.5 ppm. These glutamate peaks correspond to protons at different carbon positions of the glutamate residue within the glutathione tripeptide structure. The presence of these glutamate peaks in the GSH spectrum but not in the NAC spectrum reflects the structural difference between the two molecules, as glutathione contains a γ-glutamate residue while NAC does not.

With continued reference to FIG. 7, the difference in echo times between the edited and unedited spectra may affect which metabolites are visible and how clearly the metabolites may be distinguished. Longer echo times, such as the 120 milliseconds used for the edited spectra, may help isolate signals from specific metabolites. However, longer echo times may result in reduced overall signal strength due to T2 relaxation effects. Shorter echo times, such as the 35 milliseconds used for the unedited spectra, may provide stronger signals but with potentially more overlap between signals from different metabolites. The spectral data shown in FIG. 7 demonstrate that both NAC and GSH produce overlapping β-Cys peaks at approximately 2.9 ppm, confirming that conventional MRS quantitation based on the cysteine β-proton signal alone may be unable to differentiate between NAC and GSH contributions to the measured signal.

The methods described herein enable optimization of NAC dosing regimens for treating various brain disorders. By separately quantifying NAC and GSH concentrations in brain tissue, clinicians may tailor dosing strategies based on the specific therapeutic targets relevant to different neurological conditions. The ability to differentiate between NAC and GSH allows for evidence-based decisions regarding dosage amounts and intervals between doses.

For Parkinson's disease, auto-oxidation of dopamine is thought to be a source of disease-relevant oxidative stress. In some cases, increased GSH content may be a relevant pharmacological target on which to design dose level and dose interval for patients with Parkinson's disease. The methods described herein may allow clinicians to monitor GSH accumulation in brain tissue following NAC administration, thereby enabling adjustment of dosing parameters to achieve target GSH concentrations. By measuring GSH independently of NAC, clinicians may determine whether administered NAC is being converted to GSH at rates sufficient to provide antioxidant protection against dopamine-related oxidative damage.

For acute concussion and traumatic brain injury, the relevant biomarker on which to optimize NAC dose level and dose interval may be a balance between NAC level and GSH level. In some cases, achieving dual antioxidant and anti-neuroexcitotoxicity activity may be desirable for treating acute concussion. NAC may provide anti-neuroexcitotoxicity effects through modulation of the cystine-glutamate antiporter system, while GSH may provide catalytic antioxidant activity. The methods described herein may enable clinicians to monitor both NAC and GSH concentrations simultaneously, allowing optimization of dosing to achieve a desired balance between these two molecular species in brain tissue.

Intravenous NAC at 150 mg/kg infused over one hour has been reported to yield time-dependent MRS signatures within the occipital cortex of human subjects. The methods described herein may be used to delineate the route, rate, and longevity by and with which exogenously administered NAC is transported, transformed into, and persists as intracranial active metabolites. By performing serial MRS measurements following NAC administration, clinicians and researchers may characterize the pharmacokinetics of NAC and NAC-derived metabolites in brain tissue. This pharmacokinetic information may inform decisions regarding dose intervals to maintain therapeutic concentrations of NAC, GSH, or both in brain tissue.

In some cases, the methods described herein may be used to evaluate different routes of administration for NAC. By comparing the temporal and spatial patterns of NAC and NAC-derived metabolites in brain tissue following different administration routes, clinicians may determine which route provides the desired pharmacokinetic profile for a given therapeutic application. The ability to separately measure NAC and GSH may reveal differences in the conversion of NAC to GSH depending on the route of administration.

In some cases, the methods described herein may be used to personalize NAC therapy for individual patients. Inter-individual variability in NAC metabolism and blood-brain barrier permeability may result in different pharmacokinetic profiles among patients receiving the same dose of NAC. By measuring NAC and GSH concentrations in brain tissue for individual patients, clinicians may adjust dosing parameters to achieve target concentrations based on each patient's metabolic characteristics. This personalization of NAC therapy may improve treatment outcomes for patients with neurological and psychiatric conditions.

The quantification techniques described herein may also serve as tools for research into NAC's mechanisms of action. By separately measuring NAC and GSH in brain tissue under different experimental conditions, researchers may elucidate which of NAC's therapeutic effects are attributable to the intact NAC molecule and which are attributable to NAC-derived GSH or other metabolites. This mechanistic understanding may inform the development of enhanced therapeutic protocols and may guide the design of future clinical trials evaluating NAC for brain disorders.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross-referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

### REFERENCES

1. Atkuri K R et al. N-acetylcysteine—a safe antidote for cysteine/glutathione deficiency. Curr Opin Pharmacol. 2007 August; 7 (4): 355-359. doi: 10.1016/j.coph.2007.04.005.i.
2. Cacciatore I et al. Prodrug Approach for Increasing Cellular Glutathione Levels Molecules 2010, 15, 1242-1264; doi: 10.3390/molecules15031242.
3. Cacciatore I et al. Recent Advances in the Treatment of Neurodegenerative Diseases Based on GSH Delivery Systems. Oxidative Medicine & Cellular Longevity. Volume 2012, Article ID 240146, 12 pages. doi: 10.1155/2012/240146.
4. Dean O et al. N-acetylcysteine in psychiatry: current therapeutic evidence and potential mechanisms of action. J Psychiatry Neurosci 2011; 36 (2): 78-86.
5. Dhouib I E et al. A minireview on N-acetylcysteine: An old drug with new approaches. Life Sciences 151 (2016) 359-363.
6. Holmay M J et al. N-acetylcysteine Boosts Brain and Blood Glutathione in Gaucher and Parkinson's Diseases. Clin Neuropharmacol. 2013, 36 (4): 103-106. doi: 10.1097/WNF.0b013e31829ae713.
7. Karuppagounder S S et al. N-Acetylcysteine Targets 5 Lipoxygenase-Derived, Toxic Lipids and Can Synergize With Prostaglandin E2 to Inhibit Ferroptosis and Improve Outcomes Following Hemorrhagic Stroke in Mice. ANN NEUROL 2018; 84:854-87.
8. Katz M et al. Cerebrospinal fluid concentrations of N-acetylcysteine after oral administration in Parkinson's disease. Parkinsonism and Related Disorders 21 (2015) 500e503.
9. Mclellan L I et al. Uptake and distribution of N-acetylcysteine in mice: tissue-specific effects on glutathione concentrations. Carcinogenesis vol. 16 no.9 pp. 2099-2106, 1995.
10. Paul B D et al. Cysteine metabolism in neuronal redox homeostasis. Trends Pharmacol Sci. 2018 May; 39 (5): 513-524. doi: 10.1016/j.tips.2018.02.007.
11. Rae CD Williams S R. Glutathione in the human brain: Review of its roles and measurement by magnetic resonance spectroscopy. Analytical Biochemistry S0003-2697 (16) 30434-1DOI: 10.1016/j.ab.2016.12.022.
12. Raftos J E et al. Kinetics of uptake and deacetylation of N-acetylcysteine by human erythrocytes. The International Journal of Biochemistry & Cell Biology 39 (2007) 1698-1706.
13. Reyes R C et al. Neuronal Glutathione Content and Antioxidant Capacity can be Normalized In Situ by N-acetyl Cysteine Concentrations Attained in Human Cerebrospinal Fluid. Neurotherapeutics (2016) 13:217-225. DOI 10.1007/s1331.1-015-0404-4.
14. Samuni Y et al. The chemistry and biological activities of N-acetylcysteine. Biochimica et Biophysica Acta 1830 (2013) 4117-4129.
15. Shahripour R B et al. N-acetylcysteine (NAC) in neurological disorders: mechanisms of action and therapeutic opportunities. Brain and Behavior 2014; 4 (2): 108-122. doi: 10.1002/brb3.208.
16. Tardiolo G et al. Overview on the Effects of N-Acetylcysteine in Neurodegenerative Diseases. Molecules 2018, 23, 3305; doi: 10.3390/molecules23123305.
17. Zhou J et al. Intravenous Administration of Stable-Labeled N-Acetylcysteine Demonstrates an Indirect Mechanism for Boosting Glutathione and Improving Redox Status. Journal of Pharmaceutical Sciences, Vol. 104, 2619-2626 (2015).
18. Rushworth, G et al. Existing and potential therapeutic uses for N-acetylcysteine: the need for conversion to intracellular glutathione for antioxidant benefits. Pharmacol Ther. 2014 February;141 (2): 150-9. doi: 10.1016/j.pharmthera.2013.09.006. Epub 2013 Sep. 28. PMID: 24080471.
19. Sanaei Nezhad F, Anton A, Parkes L M, Deakin B, Williams S R. Quantification of glutathione in the human brain by MR spectroscopy at 3 Tesla: Comparison of PRESS and MEGA-PRESS. Magn Reson Med. 2017 October;78 (4): 1257-1266. doi: 10.1002/mrm.26532. Epub 2016 Oct. 31. PMID: 27797108; PMCID: PMC5469715.

1. A method for separately quantifying N-acetylcysteine and glutathione in brain tissue, comprising:
administering N-acetylcysteine to a subject;
acquiring a first magnetic resonance spectroscopy signal from a region of brain tissue of the subject, the first magnetic resonance spectroscopy signal comprising a cysteine β-proton signal;
acquiring a second magnetic resonance spectroscopy signal from the region of brain tissue, the second magnetic resonance spectroscopy signal comprising an N-acetyl proton signal; and
determining a concentration of N-acetylcysteine in the region of brain tissue based on the N-acetyl proton signal and determining a concentration of glutathione in the region of brain tissue based on a difference between the cysteine β-proton signal and the N-acetyl proton signal.

2. The method of claim 1, wherein the cysteine β-proton signal resonates at approximately 2.95 parts per million.

3. The method of claim 1, wherein the N-acetyl proton signal resonates at approximately 2.0 parts per million.

4. The method of claim 1, wherein acquiring the first magnetic resonance spectroscopy signal and acquiring the second magnetic resonance spectroscopy signal are performed using a magnetic resonance imaging device operating at 3.0 Tesla.

5. The method of claim 1, wherein the region of brain tissue comprises a voxel within an occipital cortex of the subject.

6. The method of claim 1, wherein administering N-acetylcysteine to the subject comprises intravenously infusing N-acetylcysteine at a dose of 150 mg/kg over one hour.

7. The method of claim 1, further comprising acquiring a baseline magnetic resonance spectroscopy signal from the region of brain tissue prior to administering N-acetylcysteine to the subject.

8. The method of claim 7, wherein determining the concentration of glutathione in the region of brain tissue comprises comparing the cysteine β-proton signal acquired after administering N-acetylcysteine with the baseline magnetic resonance spectroscopy signal.

9. The method of claim 1, wherein acquiring the first magnetic resonance spectroscopy signal and acquiring the second magnetic resonance spectroscopy signal comprises using spectral editing with a pulse sequence technique to isolate the cysteine β-proton signal and the N-acetyl proton signal.

10. A method for optimizing a dosing regimen of N-acetylcysteine for treatment of a brain disorder, comprising:
administering a dose of N-acetylcysteine to a subject having the brain disorder;
performing magnetic resonance spectroscopy on brain tissue of the subject to acquire spectral data comprising a cysteine β-proton resonance signal and an N-acetyl proton resonance signal;
separately quantifying a concentration of N-acetylcysteine and a concentration of glutathione in the brain tissue based on the spectral data, wherein the concentration of N-acetylcysteine is determined based on the N-acetyl proton resonance signal and the concentration of glutathione is determined based on a comparison of the cysteine β-proton resonance signal and the N-acetyl proton resonance signal; and
adjusting at least one of a dosage amount or a dosing interval of N-acetylcysteine based on the separately quantified concentrations of N-acetylcysteine and glutathione.

11. The method of claim 10, wherein the brain disorder comprises Parkinson's disease.

12. The method of claim 11, wherein adjusting at least one of the dosage amount or the dosing interval comprises increasing the dosage amount or decreasing the dosing interval to achieve a target glutathione concentration in the brain tissue.

13. The method of claim 10, wherein the brain disorder comprises traumatic brain injury.

14. The method of claim 13, wherein adjusting at least one of the dosage amount or the dosing interval comprises achieving a balance between the concentration of N-acetylcysteine and the concentration of glutathione in the brain tissue to provide antioxidant activity and anti-neuroexcitotoxicity activity.

15. The method of claim 10, further comprising performing magnetic resonance spectroscopy on the brain tissue of the subject at a plurality of time points following administering the dose of N-acetylcysteine to characterize pharmacokinetics of N-acetylcysteine and glutathione in the brain tissue.

16. The method of claim 10, wherein separately quantifying the concentration of N-acetylcysteine and the concentration of glutathione comprises subtracting a contribution of the N-acetyl proton resonance signal from the cysteine β-proton resonance signal to isolate a glutathione-specific signal.

17. A system for quantifying N-acetylcysteine and glutathione in brain tissue, comprising:
a magnetic resonance imaging device configured to perform magnetic resonance spectroscopy on a region of brain tissue of a subject who has been administered N-acetylcysteine; and
a processor configured to:
receive magnetic resonance spectroscopy data from the magnetic resonance imaging device, the magnetic resonance spectroscopy data comprising a cysteine β-proton signal and an N-acetyl proton signal from the region of brain tissue;
determine a concentration of N-acetylcysteine in the region of brain tissue based on the N-acetyl proton signal; and
determine a concentration of glutathione in the region of brain tissue based on a subtraction of a contribution of N-acetylcysteine from the cysteine β-proton signal.

18. The system of claim 17, wherein the magnetic resonance imaging device operates at 3.0 Tesla and is configured to acquire the magnetic resonance spectroscopy data using spectral editing with a pulse sequence technique.

19. The system of claim 18, wherein the processor is further configured to receive baseline magnetic resonance spectroscopy data acquired prior to administration of N-acetylcysteine to the subject and to determine changes in the concentration of N-acetylcysteine and the concentration of glutathione relative to the baseline magnetic resonance spectroscopy data.

20. The system of claim 17, wherein the cysteine β-proton signal resonates at approximately 2.95 parts per million and the N-acetyl proton signal resonates at approximately 2.0 parts per million.
